(19) United States
(12) Patent Application Publication
(10) Pub. No.: US 2019/0307715 A1
(43) Pub. Date: Oct. 10, 2019

(54) PREVENTION AND/OR TREATMENT OF NEURODEGENERATIVE DISEASE

(71) Applicant: Howard Foundation Holdings Ltd, Cambridgeshire (GB)

(72) Inventors: Alan Norman HOWARD, Cambridgeshire (GB); John NOLAN, Waterford (IE); Riona MULCAHY, Waterford (IE)

(73) Assignee: Howard Foundation Holdings Ltd, Cambridgeshire (GB)

(21) Appl. No.: 16/426,771

(22) Filed: May 30, 2019

Related U.S. Application Data
(63) Continuation-in-part of application No. 15/980,083, filed on May 15, 2018.

(30) Foreign Application Priority Data
Dec. 4, 2017 (GB) 1720119.5

(51) Int. Cl. A61K 31/202; A61K 31/047; A61K 31/355; A61K 9/00; A61K 9/20; A61K 9/48; A61P 25/28

(52) U.S. Cl. CPC A61K 31/202; A61K 31/047; A61K 31/355; A61P 25/28; A61K 9/20; A61K 9/48; A61K 9/0053

(57) ABSTRACT

A method for the prevention and/or treatment of mild cognitive impanment in a human subject; the method comprising the step of administering an effective amount of a composition comprising each of the following: lutein, zeaxanthin, meso-zeaxanthin, vitamin E, and at least one omega-3 fatty acid.

### RELATED APPLICATIONS

This application is a continuation-in-part of U.S. application Ser. No. 15/980,083, filed May 15, 2018 in the U.S. Patent and Trademark Office, which claims the benefit of priority to United Kingdom Application No. 1720119.5, filed Dec. 4, 2017, in the Intellectual Property Office of the United Kingdom. All disclosures of the document named above are incorporated herein by reference.

### FIELD OF THE INVENTION

The present invention relates to the use of combinations of substances for the prevention and/or treatment of neurodegenerative disease, especially dementia, in human subjects, and more especially prevention and/or treatment of mild cognitive impairment (“MCI”) and/or prevention of Alzheimer's disease.

### BACKGROUND OF THE INVENTION

Alzheimer's disease (AD) is the most common type of dementia, followed by vascular dementia. In the UK, the number of people with AD is 850,000 and in the USA is 5.5 million With increasing age and population, it is anticipated that the overall prevalence of the disease will continue to increase [ref. 1]. Despite considerable research effort, the cause of the disease is still unknown but established risk factors include age, family history of disease and education [ref. 1] and putative risk factors include cigarette smoking, physical inactivity and social isolation [ref. 32]. The effectiveness of current pharmacological treatments (medications) is strictly limited and varies among individuals. Moreover, the effect of current medications is at best only palliative, as they cannot halt disease progression.

The UK NHS suggests, as preventative, the following: cessation of smoking, limiting alcohol consumption, a healthy well balanced diet, staying physically and mentally active.

There have been numerous studies on the effects of the Mediterranean Diet, a diet characterised by a high intake of vegetables, olive oil, and a moderate intake of fish, dairy products and wine. There is a consensus that adherence to such diets is associated with better cognitive performance [refs. 2-7] and a reduced risk of dementia, especially AD [refs. 8-12], but no clear evidence of the exact foodstuffs responsible.

Some studies have pointed to an effect from omega-3 fatty acids, of which docosahexaenoic acid (DHA) is found in high concentration in fish oil and in fatty fish such salmon and herring. There is a substantial concentration of DHA in the human brain where it forms a structural component within this neural tissue. High consumption of omega-3 fatty acids is associated with better cognitive performance [ref. 13] and a reduced risk of dementia [refs. 14-17]. However, interventional studies have shown conflicting results, with some demonstrating improvements in cognition [refs. 18, 19] and others demonstrating no beneficial effect [refs. 20, 21].

Other food items of interest include the carotenoids lutein (L) and zeaxanthin (Z). These carotenoids, which are dietary of origin are found in certain fruits and vegetables (e.g. spinach, broccoli, peppers, melon) [ref. 22]. In humans, L and Z are found in high concentrations in the eye (specifically the centre part of the retina known as the macula, where they are referred to as “macular pigment” or “macular carotenoids”) and brain [refs. 23, 24]. High carotenoid intake has been found to result in a reduced risk of AD [refs. 25,-27]. Some studies administering L and Z have shown improvement in different domains of cognition [refs. 28, 29], while another randomised trial showed no benefit [ref. 30].

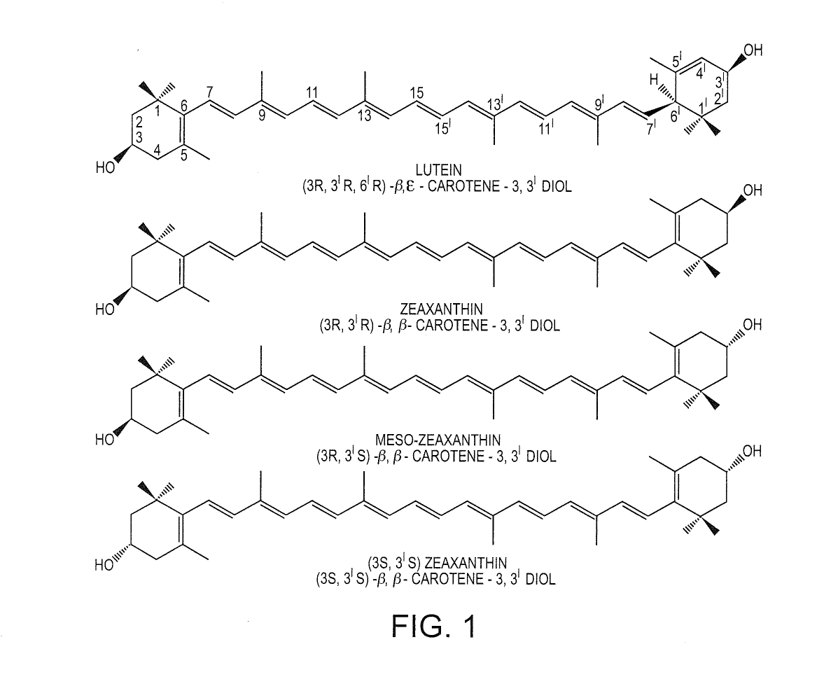

Another macular carotenoid is meso-zeaxanthin (MZ), which is a stereoisomer of zeaxanthin. The chemical structures of L, Z and MZ are shown in FIG. 1.

Vitamin E is also present in the brain, and high plasma concentrations of vitamin E have been associated with a reduced risk of AD [ref. 31]. There have been no reports of successful treatment of AD following administration of this vitamin.

In summary, there is general agreement that there are substances in the brain which play a role in preventing AD, but attempts so far to identify and/or use them have been unsuccessful.

Compositions comprising all three macular carotenoids (L, Z and MZ) are commercially available as nutritional supplements. One example of such a supplement is sold under the trade mark Macushield®, and consists of capsules containing the three macular carotenoids L, Z and MZ in the amounts of 10 mg, 2 mg and 10 mg, respectively, per capsule. WO 2013/005037 discloses the use of such a composition for improving the visual performance of a human subject, the composition optionally further comprising a fish oil and/or an omega-3 fatty acid.

US2016/0067203 (Lion Corporation) discloses and claims compositions for improving cognitive function, the compositions comprising docosahexaenoic acid (DHA) in combination with lutein, zeaxanthin and capsanthin.

The specification includes the results of a “Passive Avoidance Test” performed using mice fed the test composition or a control composition for 3 months (see especially Example 5 in Table 2 of the prior art document). The mice were given a mild electric shock when they went into a darkened compartment, and learned to avoid the darkened compartment when the test was repeated. The prior art specification also presents data (Table 3) suggesting that the amount of amyloid protein in the brain was reduced in the test mice compared to the controls (although the numbers of animals involved in this experiment were very small, n=3 and n=4), so these results are not statistically significant. Moreover, there are no data from experiments with human subjects. Also, there is no evidence that the exemplified to composition could improve cognitive function in, or stabilise the condition of, subjects already suffering from cognitive impairment.

WO2006/116755 (Trustees of Tufts College) discloses and claims a composition having “synergistic amounts of lutein and DHA for use in improvement of cognitive function”. The claims are based on a trial in which 50 human subjects (all female) were given daily supplements, over a 4 month period, containing (i) a placebo; or (ii) DHA or lutein; or (iii) DHA and lutein in combination. Various cognitive function tests were performed at start and end of the period of supplementation, and the results are shown in Table 2 of the prior art document. All compositions, except the placebo, gave statistically significant (<0.05) improvements in a verbal fluency test (hence, there was no evidence of any synergy between the DHA and lutein).

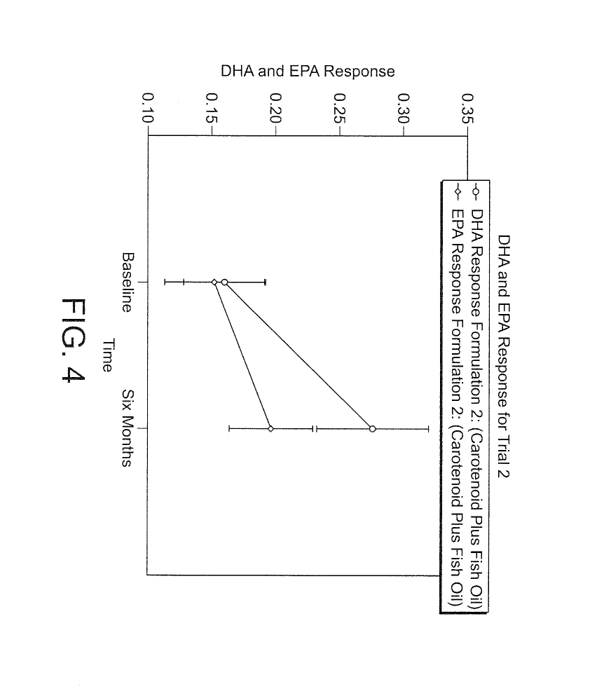

In addition however, subjects given the DHA/lutein combination demonstrated statistically significant improvements in a Shopping List Memory Test and an MIR (“Memory-in-Reality”) Apartment Test. However for the latter, it is clear from FIG. 4 of the prior art publication that the baseline score was unusually low for that group, so a big improvement was perhaps nothing to do with the supplementation. Finally, there is no evidence to show that the DHA/lutein combination could have any positive effect on subjects already experiencing cognitive impairment.

### SUMMARY OF THE INVENTION

The inventors have found that the macular carotenoids such as a mixture of meso-zeaxanthin (MZ), lutein (L) and zeaxanthin (Z), or omega-3 fatty acids, given separately, have no effect individually on the progression of dementia, but when given together, in combination with vitamin E, are remarkably and surprisingly effective in halting or retarding the progression of mild cognitive impairment (“MCI”) and improving cognitive function.

Accordingly, in a first aspect, the present invention provides a method for the prevention and/or treatment of mild cognitive impairment in a human subject; the method comprising the step of administering an effective amount of a composition comprising each of the following: lutein, zeaxanthin, meso-zeaxanthin, vitamin E, and at least one omega-3 fatty acid.

In the first aspect of the invention the composition may be administered by any suitable route to a human subject including, for example, intra-venous administration. Preferably however the composition is administered orally, and advantageously the composition is formulated so as to be suitable for oral administration.

In one embodiment, preventing and/or treating mild cognitive impairment in a human subject is assessed by measuring performance of the subject in the immediate memory test of the Repeatable Battery for the Assessment of Neuropsychological Status (RBANS) (as defined in Randall et al., Journal of Clinical and Experimental Neuropsychology 1998; 20(3) 310-319). More especially, successfully preventing and/or treating MCI in a human subject will cause, over a 12 month period, an improvement in the subject's RBANS immediate memory score, typically an improvement of at least 5%, preferably an improvement of at least 10%, and most preferably an improvement of at least 15%, relative to the subject's RBANS immediate memory score at the commencement of the 12 month period.

In some embodiments of the invention the subject will be cognitively intact (i.e. not exhibit any symptoms of MCI) at the start of the period of treatment. A cognitively intact subject is defined as having an RBANS immediate memory index score of 85 or higher.

In other embodiments of the invention, a subject may be suffering from MCI at the start of the period of treatment. A subject is considered to be suffering from MCI if they have an RBANS immediate memory index score of 80 or less, and an MMSE score of 26 or higher.

Generally, untreated subjects with MCI may experience a deterioration of their cognitive performance over time, and improvement in their cognitive function is most unusual. Hence, the method of the present invention, which can bring about an improvement in cognitive function, is most unexpected and to be welcomed.

In addition, it has been found that subjects with MCI are at increased risk of progressing to Alzheimer's disease over time than those without MCI (Petersen et al., 2001 Archives of Neurology 58:(12) 1985-1992). Accordingly, in some embodiments of the present invention, by preventing or delaying the symptoms of MCI in a subject, it is possible to prevent or delay the progression of that individual to Alzheimer's disease.

The composition may be administered to a human subject who is not yet exhibiting any symptoms of MCI or dementia (e.g. Alzheimer's disease), with a view to preventing the subject from developing MCI or dementia, or with a view to deferring the time at which the subject starts to exhibit one or more of the symptoms of MCI or dementia, (e.g. Alzheimer's disease). Either of these may be regarded as “preventing” MCI or dementia or, specifically, Alzheimer's disease—in the first instance indefinitely, and in the second instance for at least a period of time. The period of time for which the onset of one or more of the symptoms of MCI or dementia/Alzheimer's disease may be deferred may depend on the age of the subject when the composition is administered, and on other factors which influence the susceptibility of the subject to dementia (e.g. genotype, diet, smoking history etc.). Preferably the onset of the one or more symptoms of MCI or dementia is deferred for at least 6 months, more preferably at least 12 months, and most preferably at least 18 months. In particular, the one or more symptoms of dementia typically comprise or consist of cognitive impairment. The inventors believe that administration of the macular carotenoids, vitamin E, and one or more omega-3 polyunsaturated fatty acids (especially DHA and/or eicosapentaenoic acid, EPA) in sufficient amounts, to a human subject who is not yet exhibiting any symptoms of Alzheimer's disease, could prevent the occurrence of Alzheimer's disease in the subject indefinitely.

Alternatively, the composition may be administered to a subject who is already exhibiting one or more symptoms of MCI or dementia (especially Alzheimer's disease), with a view to preventing or retarding the progression of the disease. Preventing the progression of the disease means that the severity of the existing one or more symptoms of MCI or dementia does not increase. In particular, preventing the progression of the disease will preferably comprise or consist of substantially preventing progression of cognitive impairment in the subject. Retarding the progression of the disease means that the severity of the symptoms increases more slowly than would have been the case if intervention (i.e. administering the composition of the invention) had not occurred. Preventing or retarding the progression of dementia (e.g. Alzheimer's disease) is regarded as treating the disease for present purposes.

Diagnosing dementia is known to be challenging. A useful diagnostic aid is the mini mental state examination or “MMSE” (Tombaugh & McIntyre 1992 J. Am. Geriatr. Soc. 40, 922-935). For present purposes, a subject can be considered to have dementia if they obtain an MMSE score of 25 or less and where any other causes of cognitive impairment (e.g. head injury, chronic alcohol abuse, fever, urinary tract infections etc.) can be excluded.

Preventing progression of cognitive impairment in a subject, as a result of administering the composition in accordance with the invention, may be checked by performing a mini mental state examination. In some embodiments, a subject already suffering from dementia (i.e. having an MMSE score of 25 or less) and consuming a composition in accordance with the invention will experience a reduction in MMSE score of no more than 2 points over an 18 month period, more preferably no more than 1 point over an 18 month period, and most preferably a zero point reduction over an 18 month period.

As noted previously, the composition comprises at least one omega-3 fatty acid. For present purposes, unless the context dictates otherwise, the term “fatty acid” is intended also to encompass not only the free acid but also derivatives of fatty acids, such derivatives encompassing, in particular, esters, especially esters formed with glycerol (monoglycerides, diglycerides and, preferably, triglycerides), and salts. Preferred salts are those containing monocations, such as Na⁺, K⁺ or NH₄⁺. Most preferred salts are those comprising metallic monocations. The free fatty acid or the triglyceride is the most preferred form of the compound.

The omega-3 fatty acid component of the composition preferably comprises an omega-3 polyunsaturated fatty acid or derivative thereof, most preferably docosahexaenoic acid (DHA) or a derivative thereof. The composition may contain two or more omega-3 fatty acids. The composition may comprise eicosapentaenoic acid (EPA). In one embodiment the composition comprises both DHA and EPA.

A convenient source of omega-3 fatty acids is fish oil. Accordingly, in a preferred embodiment, the composition comprises fish oil. Since fish oil has quite a strong odour, it may be preferred to use deodourised fish oil, which is commercially available. Another source of omega-3 fatty acids is nut oil. Without being bound by any particular theory, the inventors believe that DHA is the most active omega-3 fatty acid in terms of preventing and/or treating MCI or dementia. Nut oil does not contain substantial amounts of DHA and therefore is not preferred for the purposes of the present invention.

Other sources of fatty acids include algae (see Ji et al, 2015 “Omega-3 Biotechnology: A green and sustainable process for omega-3 fatty acids production” Front Bioeng. Biotechnol. 3, 158).

The composition may be formulated in a ‘bulk’ form, to be admixed, for example, with a conventional foodstuff, including dairy foodstuffs (e.g. incorporated into butter or ice cream) or non-diary foodstuffs (e.g. margarine, vegetable stock or fish-stock preparations). More preferably however the composition is formulated in unit dosage form, preferably one suitable for oral consumption by a human subject, including a tablet, capsule, gel, liquid, powder or the like. The one or more macular carotenoids may be granulated, for example as microcapsules, before inclusion in the formulation.

Conveniently, but not necessarily, the composition may be packaged in a foil blister pack, of the sort known to those skilled in the art. Desirably one or two of the doses are taken each day, the amount of active agents in the doses being adjusted accordingly.

The composition may conveniently comprise conventional diluents, especially vegetable oils such as sunflower, safflower, corn oil and rape seed oils, excipients, bulking agents and the like which are well known to those skilled in the art. Such substances include, calcium and/or magnesium stearate, starch or modified starch.

Other conventional formulating agents may be present in the composition, including any one or more of the following non-exhaustive list: acidity regulators; anticaking agents (e.g. sodium aluminosilicate, calcium or magnesium carbonate, calcium silicate, sodium or potassium ferricyanide), antioxidants (e.g. vitamin C, polyphenols), colorings (e.g. artificial colorings such as FD&C Blue No. 1, Blue No. 2, Green No. 3, Red No. 40, Red No. 3, Yellow No. 5 and Yellow No. 6; and natural colorings such as caramel, annatto, cochineal, betanin, turmeric, saffron, paprika etc.); color retention agents; emulsifiers; flavours; flavour enhancers; preservatives; stabilizers; sweeteners and thickeners.

Other optional ingredients of the composition include vitamins and/or minerals. Preferably the composition comprises at least one B vitamin.

Typically the composition is administered at least once a week, preferably at least twice a week, more preferably three times a week, and most preferably daily. In a typical embodiment at least one unit dosage form of the composition is taken on a daily basis. The person skilled in the art will appreciate that the frequency of consumption can be adjusted to take account of the concentration of active agents (vitamin E, the macular carotenoids, omega-3 fatty acid), present in the formulation. The administration of the composition can be adjusted accordingly.

Preferably, in order to prevent the onset of one or more symptoms of dementia, the composition should be administered to subjects at or before the age of 50, before which age the onset of dementia is rather rare.

In order to obtain a preventative effect, the composition should preferably be administered, typically at least 2 or 3 times a week, or daily, over a period of at least 6 months, preferably over a period of at least 12 months, and even more preferably over a period of at least 18 months. For treatment of patients already exhibiting one or more symptoms of dementia, the composition should preferably be administered indefinitely, for as long as the patient is able to take the composition.

For present purposes the “active agents” in the composition are considered to be vitamin E, the macular carotenoids and the at least one omega-3 fatty acid.

The precise concentration of the active agents in the composition of the invention is not critical: a beneficial effect on the subject can be obtained by consuming larger doses of a composition comprising lower concentrations of active agents, or vice versa.

The respective ratio in the composition of the macular carotenoids is not thought to be critical and can vary quite widely. For example, the percentage of either MZ or lutein in the composition can range from 10% to 90% (of the macular carotenoid present in the composition). The percentage of zeaxanthin can typically range from about 5 to 45% (of the macular carotenoid present in the composition). One particular composition has an MZ:lutein:zeaxanthin ratio of 10:10:2 (or 45%, 45%, 10%), but this is not essential. Another typical composition has a ratio (MZ:L:Z) of 12:10:2.

One preferred composition for use in performance of the invention is in unit dosage form, with each unit dose comprising 5 mg meso-zeaxanthin, 5 mgs lutein, 1 mg zeaxanthin, 7.5 mgs vitamin E and and fish oil (preferably 0.5 gm of fish oil).

Another preferred composition for use in performance of the invention is in unit dosage form with each unit dose comprising 7.5 mgs vitamin E, 7.5 mgs meso-zeaxanthin, 2.5 mgs lutein, 0.5 mg zeaxanthin and fish oil (preferably 0.5 gm fish oil).

Desirably an average daily dose of the composition provides a total macular carotenoid content of up to, but not exceeding, 100 mg, preferably up to, but not exceeding, 75 mg, and most preferably up to, but not exceeding, 50 mg. Desirably the average daily dose of the composition provides a minimum total macular carotenoid content of at least 18 mg, more preferably at least 20 mg, and most preferably at least 22 mg. Such concentrations are known to be well-tolerated with substantially no adverse effects.

Advantageously, the average daily dose of the composition provides an amount of omega-3 fatty acid in the range 10 mg to 2 gms, more preferably in the range 20 mgs to 2 gms, and most preferably in the range 25 mgs to 1 gram.

The foregoing omega-3 fatty acid content may be provided entirely by DHA, or by a combination of two or more omega-3 fatty acids, one of which is preferably DHA. DHA desirably constitutes at least 50% of the omega-3 fatty acid content of the composition, preferably at least 55%, more preferably at least 60%, and most preferably at least 65%.

Advantageously, the average daily dose of the composition provides an amount of vitamin E in the range 2 mgs to 100 mgs, more preferably in the range 5-50 mgs, and most preferably in the range 5 to 25 mgs.

The invention will now be further described by way of illustrative example and with reference to the accompanying drawings, in which:

FIG. 1 is a schematic representation of the structural formulae for lutein, zeaxanthin and meso-zeaxanthin;

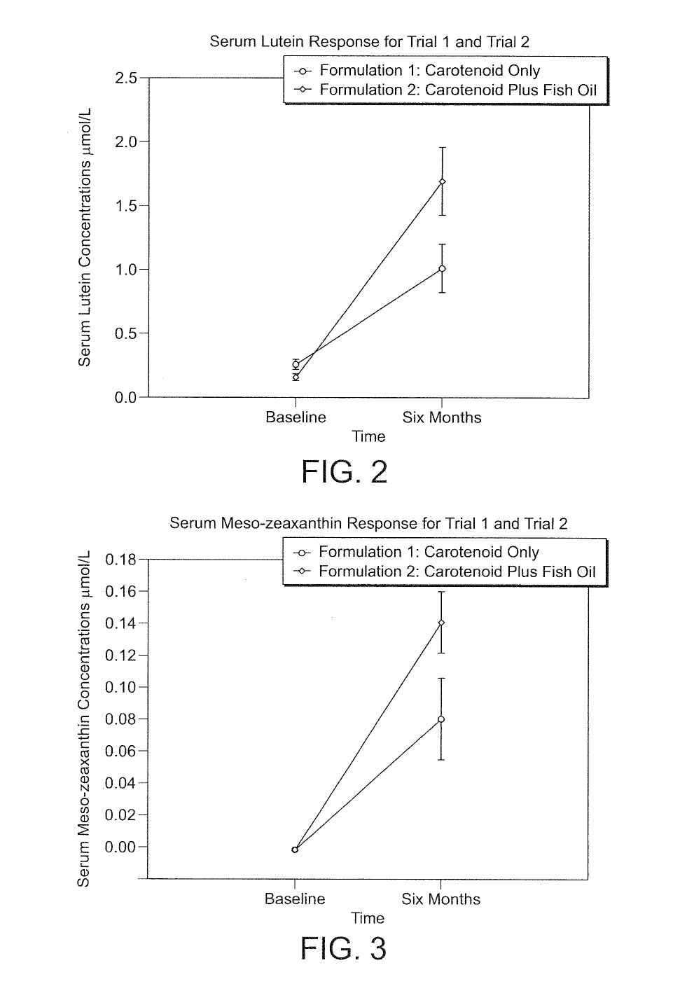

FIG. 2 is a graph of serum lutein concentration (μmol/L) against time (months);

FIG. 3 is a graph of serum meso-zeaxanthin concentration (μmol/L) against time (months);

FIG. 4 is a graph of either DHA or EPA response (arbitrary units) against time (months);

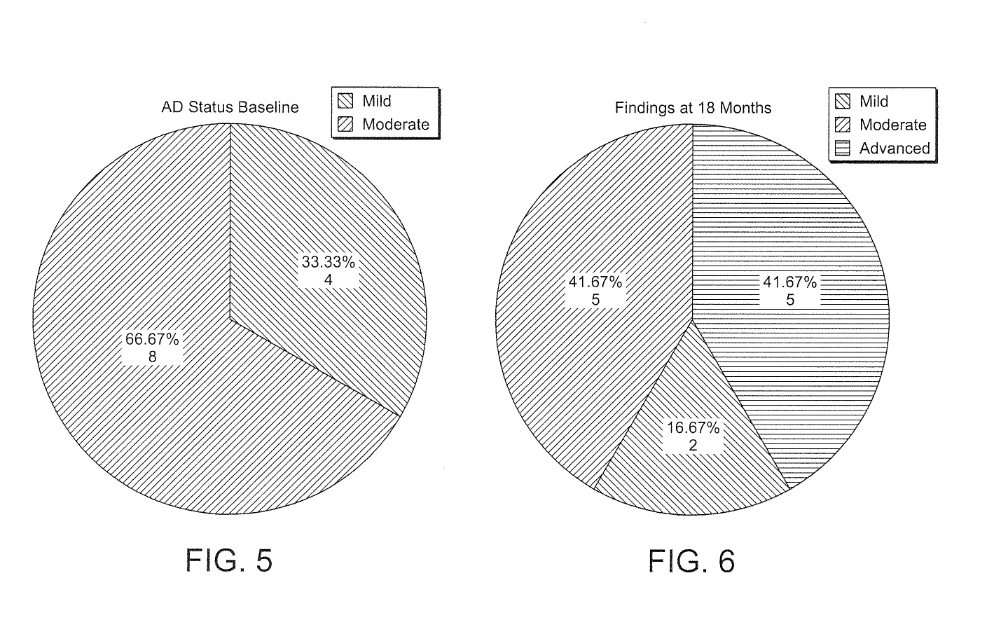

FIGS. 5-8 are pie charts illustrating the severity of dementia symptoms in patients at 0 months or at 18 months; and

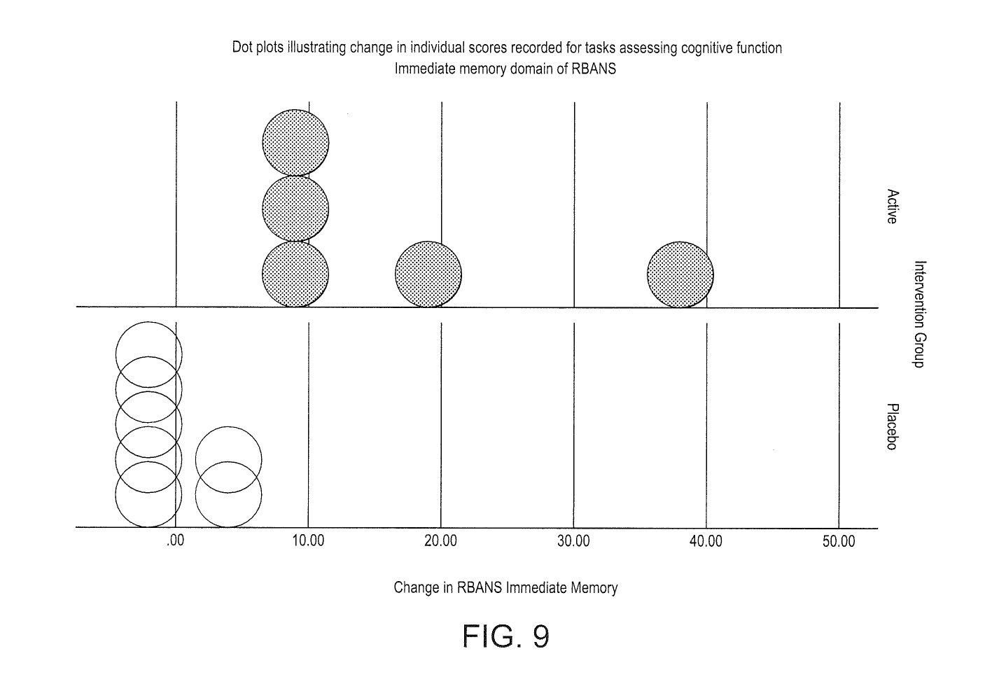

FIG. 9 is a dot plot illustrating the change in the RBANS immediate memory test scores of individual subjects following supplementation with a composition according to the method of the invention (shaded symbols) or a placebo (unshaded symbols).

### EXAMPLES

### Example 1

This example relates to a scientific research study conducted at the Nutrition Research Centre Ireland (NRCI).

Patients with AD were recruited from the University Hospital Waterford (UHW) via the Age-Related Care Unit department. This study was conducted in accordance with full sensitivity to the ethical requirements of the patients recruited. The study objectives and methodology complied fully with the widely-recognized international text and codes of practice, such as the Declaration of Helsinki and Good Research Practice. A protocol was developed specifically for this study to ensure that informed consent was obtained appropriately, and in keeping with the ethical code germane to obtaining consent from vulnerable subjects such as patients with AD.

In brief, the inventors performed three trial experiments. Two trials were conducted in patients with AD and one trial experiment in age-matched control subjects (free of AD).

In trial group 1, patients with AD (n=12) were given a daily dietary supplement containing macular carotenoids, but no omega-3 fatty acid. The supplement took the form of a capsule of a commercially-available preparation (“Macushield”™). Each capsule comprises approximately 10 mg lutein (“L”), 10 mg meso-zeaxanthin (“Z”) and 2 mg zeaxanthin (“Z”). One capsule per day was administered, under the supervision of the patients' carers, over a period of 18 months.

In trial group 2 (n=13) AD patients took a daily dietary supplement essentially identical to that used in trial group 1, but additionally containing 1 gm of fish oil (containing approximately 430 mg DHA and 90 mg EPA). The fish oil was of high quality and obtained from Epax® (Epax Norway AS, Alesund, Norway). Again, consumption of the supplement was overseen by the patients' carers, and continued over a period of 18 months.

The third trial group (n=31) were age-matched controls with no evidence of dementia. This group did not consume any dietary supplement.

All subjects were assessed at the start of the trial to provide a baseline. Characteristics assessed included, depending on the trial group, some/most of the following:

(i) serum concentration of L, Z and MZ;

(ii) macular pigment (MP) concentration in the macula, at 0.23 degrees of retinal eccentricity (“MP 0.23”) (for explanation see below);

(iii) MP volume (MP volume under the curve) (—see below);

(iv) LPC 22:6 and LPC 20:5 (these are measures of the phospholipid in the blood containing either DHA or EPA respectively—see below); and

(v) medical assessment of dementia (+/−“MMSE”). The baseline values for these characteristics are shown in Table 1 below.

MP was measured by dual-wavelength autofluorescence (AF) using the Spectralis HRA+OCT MultiColor instrument (Heidelberg Engineering GmbH, Heidelberg, Germany), as described by Akuffo et al., (2014 Ophthalmic Epidemiol. 21, 111-123). Pupillary dilation was performed prior to measurement and patient details were entered into the Heidelberg Eye Explorer (HEYEX) version 1.7.1.0 software. Dual-wavelength AF in this device uses two excitation wavelengths, one that is well absorbed by MP (486 nm, blue), and one that is not (518 nm, green) (Trieschmann et al., 2006, Graefes Arch. Clin. Exp. Ophthalmol. 244, 1565-1574; Dennison et al., 2013 Exp. Eye Res. 116, 190-198). The following acquisition parameters were used: high speed scan resolution, two seconds cyclic buffer size, internal fixation, 30 seconds movie and manual brightness control Alignment, focus and illumination were first adjusted in infrared mode. Once the image was evenly illuminated, the laser mode was switched from infrared to blue plus green laser light AF. Using the HEYEX software, the movie images were aligned and averaged, and a MP density map was created. MP volume was calculated as MP average times the area under the curve out to 7° eccentricity.

LPC 22:6 and LPC 20:5 assays were performed as follows: samples were extracted and analysed as previously published (Koulman, et al. 2014. “The development and validation of a fast and robust dried blood spot based lipid profiling method to study infant metabolism”. Metabolomics 10:1018-25). All samples were infused into an Exactive Orbitrap (Thermo, Hemel Hempstead UK), using a Triversa Nanomate (Advion, Ithaca US).

TABLE 1
Demographics and health variables for patients in Trials 1-3 at baseline

| | Trial 1 (n = 12) | Trial 2 (n = 13) | Trial 3 (n = 31): |
|---|---|---|---|
| Demographics and health variables | AD | AD | Controls |
| Age (years) | 78.5 ± 8.754 | 78.77 ± 7.65 | 76 ± 6.6 |
| Sex (% females) | 6 (50%) | 8 (71%) | 13 (42%) |
| Serum lutein (μmol/L) | 0.261 ± 0.142 | 0.154 ± 0.084 | 0.297 ± 0.179 |
| Serum zeaxanthin (μmol/L) | 0.048 ± 0.035 | 0.062 ± 0.031 | 0.074 ± −0.179 |
| MMSE | 19 ± 2.89 | 16 ± 2.873 | 29 ± 0.179 |
| Category Mild AD | 4 (33%) | 2 (15%) | na |
| Category Moderate AD | 8 (67%) | 10 (77%) | na |
| Category Severe AD | 0 (0%) | 1 (8%) | na |
| MP 0.23 | 0.41 ± 0.24 | — | 0.57 ± 0.17 |
| MP Volume | 4114 ± 2308 | — | 6326 ± 2258 |
| LPC 22:6 (phospholipid containing | — | 0.160 ± 0.118 | |
| DHA) | | | |
| LPC 20:5 (phospholipid containing | — | 0.153 ± 0.144 | |
| EPA) | | | |

MMSE = Mini Mental State Examination;
MP 0.23 = macular pigment at center (0.23 degrees of retinal eccentricity);
MP volume = macular pigment volume under the curve.
LPC 22:6 (phospholipid containing DHA) relative to the total lipid signal in blood. This is an excellent biomarker of DHA.
LPC 20:5 phospholipid containing EPA) = phospholipid containing EPA relative to the total lipid signal in blood. This is an excellent biomarker of EPA.

Method of Assessment

Biochemical Response:

For biochemical assessment, samples were obtained at baseline and also after 6 months of supplementation. Serum MZ, L and Z concentrations were assessed by high performance liquid chromatography (HPLC) of blood plasma. Markers of DHA and EPA were assessed by liquid chromatography (LC-) mass spectrometry (MS) metabolomics and lipidomics analysis of blood samples, using previously validated methods as detailed above.

MMSE:

This was performed under the supervision of a consultant clinician and is a validated technique. The results of the MMSE were used to guide the diagnosis of AD during patient recruitment. AD status was defined as follows: MMSE score 0-10=severe AD; MMSE score 11-20=moderate AD; MMSE score 21-25=mild AD; 26⁺=no AD. In addition to the MMSE conducted at the start of the trial, each patient was assessed for health and AD status at 18 months where possible.

### Results

### Macular Carotenoid Levels

Firstly, it should be noted that the average serum lutein concentration in Trial 2 subjects (0.154±0.084 μmol/L) was considerably lower than in the control group (0.297±0.179)—this difference was statistically significant (p=0.010) as determined by the Independent Samples T-test.

FIG. 2 shows the mean serum lutein concentrations at the start of the trial and after 6 months for trial group 1 (filled circles) and trial group 2 (empty circles). It is readily apparent the serum lutein concentration increased over the 6 month period for both groups. However, the group 2 subjects (receiving the MC and fish oil supplement) exhibited a markedly superior increase in serum lutein, which was statistically significant (p=0.002).

A similar picture was found for serum levels of MZ in the trial group 1 and 2 subjects (see FIG. 3): both groups exhibited a clear increase in serum MZ at 6 months compared to baseline; but the trial group 2 subjects displayed a greater increase than group 1 subjects, which was again statistically significant (p=0.06).

Omega 3 Fatty Acid Response

The results for the serum DHA (filled circles) and serum EPA (empty circles) are shown in FIG. 4 for the trial group 2 subjects at baseline and after 6 months. It can be seen that the EPA concentration increased, and the concentration of DHA increased quite markedly. The greater response in DHA levels was to be expected, as the fish oil supplement used in the trial contained more DHA than EPA.

Medical Response

Patient medical assessment was performed at baseline and after 18 months of supplementation. Medical assessment was performed by a qualified AD nurse under the supervision of a medical consultant. Also, the patient carer was interviewed after 18 months of the patient being on the supplement. AD status at baseline was confirmed by the medical consultant.

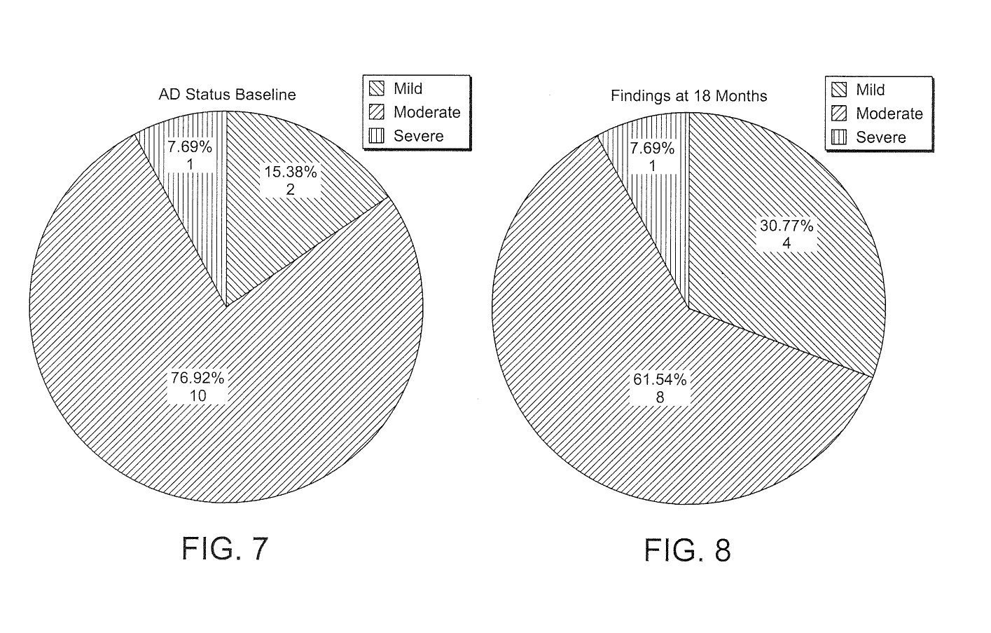

FIGS. 5 and 6 show the AD status for the trial group 1 subjects at baseline (FIG. 5) and at 18 months (FIG. 6). FIGS. 7 and 8 show the AD status for the trial group 2 subjects at baseline (FIG. 7) and at 18 months (FIG. 8).

In addition, the individual subject data for the two trial groups are presented in Table 2.

TABLE 2
Individual subject data

| | | | Sex | Baseline | Baseline | Baseline | 6-months |
|---|---|---|---|---|---|---|---|
| | Subject | Age | (M—male; | MMSE | AD | Serum L | Serum L |
| Study | ID | (years) | F = Female) | score | Status | (μmol/L) | (μmol/L) |
| Trial 1 | ADCD2 | 78 | M | 16 | Moderate | 0.072 | 0.666 |
| | ADCD3 | 81 | M | 17 | Moderate | 0.432 | 0.933 |
| | ADCD4 | 84 | M | 18 | Moderate | 0.302 | 0.973 |
| | ADCD5 | 82 | F | 23 | Mild | 0.466 | 0.676 |
| | ADCD8 | 90 | F | 15 | Moderate | 0.149 | 0.564 |
| | ADCD9 | 76 | M | 22 | Mild | 0.325 | 1.631 |
| | ADCD11 | 68 | F | 19 | Moderate | 0.397 | 0.941 |
| | ADN24 | 68 | M | 23 | Mild | 0.149 | 0.285 |
| | ADN25 | 64 | F | 15 | Moderate | 0.202 | 0.771 |
| | ADN26 | 74 | F | 21 | Mild | 0.364 | 1.972 |
| | ADN29 | 87 | F | 19 | Moderate | 0.238 | 2.415 |
| | ADN31 | 90 | M | 20 | Moderate | 0.034 | 0.364 |
| Trial 2 | C3A4 | 79 | F | 12 | Moderate | 0.051 | 0.548 |
| | C3A5 | 78 | M | 22 | Mild | 0.204 | 1.733 |
| | C3A6 | 89 | F | 14 | Moderate | 0.273 | 3.599 |
| | C3A7 | 81 | F | 13 | Moderate | 0.095 | 2.515 |
| | C3A8 | 87 | M | 17 | Moderate | 0.153 | 1.021 |
| | C3A9 | 88 | F | 21 | Mild | 0.122 | 0.536 |
| | C3A10 | 71 | F | 18 | Moderate | 0.107 | 2.722 |
| | C3A12 | 79 | F | 17 | Moderate | 0.244 | 2.229 |
| | C3A13 | 77 | M | 110 | Severe | 0.064 | 1.971 |
| | C3A14 | 69 | M | 17 | Moderate | 0.074 | 0.342 |
| | C3A16 | 87 | M | 18 | Moderate | 0.123 | 0.974 |
| | C3A17 | 74 | F | 18 | Moderate | 0.315 | 1.879 |
| | C3A19 | 65 | F | 18 | Moderate | 0.182 | 1.97 |

| | | Baseline | 6-months | 6-months | |
|---|---|---|---|---|---|
| | Subject | Serum Z | Serum Z | Serum MZ | |
| Study | ID | (μmol/L) | (μmol/L) | (μmol/L) | Observations at 18-months |
| Trial 1 | ADCD2 | 0.006 | 0.062 | 0.08 | No difference reported |
| | ADCD3 | 0.046 | 0.088 | 0.044 | No difference reported |
| | ADCD4 | 0.057 | 0.099 | 0.052 | No difference reported |
| | ADCD5 | 0.133 | 0.099 | 0.034 | No difference reported |
| | ADCD8 | 0.032 | 0.056 | 0.043 | Disease advanced; patient too sick |
| | | | | | to continue study |
| | ADCD9 | 0.076 | 0.189 | 0.086 | Disease advanced; patient in a |
| | | | | | nursing home |
| | ADCD11 | 0.057 | 0.076 | 0.044 | No information available |
| | ADN24 | 0.054 | 0.041 | 0.014 | No difference reported |
| | ADN25 | 0.035 | 0.078 | 0.027 | Disease advanced; patient unable |
| | | | | | to continue study |
| | ADN26 | 0.059 | 0.248 | 0.294 | Disease advanced; patient unable |
| | | | | | to follow instruction |
| | ADN29 | 0.02 | 0.245 | 0.234 | Disease advanced; patient too sick |
| | | | | | to continue study |
| | ADN31 | 0.003 | 0.024 | 0.018 | No difference reported |
| Trial 2 | C3A4 | 0.027 | 0.057 | 0.062 | Carer reported good improvement |
| | | | | | to memory |
| | C3A5 | 0.05 | 0.145 | 0.1 | Carer reported good improvement |
| | | | | | to sight |
| | C3A6 | 0.089 | 0.312 | 0.228 | Carer reported subject was |
| | | | | | improved and manageable |
| | C3A7 | 0.041 | 0.226 | 0.153 | No difference reported |
| | C3A8 | 0.058 | 0.094 | 0.091 | No difference reported |
| | C3A9 | 0.069 | 0.082 | 0.03 | No difference reported |
| | C3A10 | 0.026 | 0.244 | 0.186 | No difference reported |
| | C3A12 | 0.075 | 0.153 | 0.152 | Carer reported good improvement |
| | | | | | to vision |
| | C3A13 | 0.044 | 0.131 | 0.178 | No difference reported |
| | C3A14 | 0.048 | 0.039 | 0.1 | No difference reported |
| | C3A16 | 0.043 | 0.058 | 0.085 | Carer reported good improvement |
| | | | | | to memory |
| | C3A17 | 0.137 | 0.065 | 0.252 | No difference reported |
| | C3A19 | 0.105 | 0.148 | 0.226 | Carer reported good improvement |
| | | | | | to vision |

As seen from the pie charts (FIGS. 5 and 6, data from Trial 1, carotenoid only intervention) and Table 2, progression of AD was evident in this group over the 18 month period, with 42% of patients' health status dropping to the point that they could not continue in the trial. Reasons for dropout include: patient moved into a nursing home due to AD progressing; patient became too unwell to continue; cognitive decline too severe; no longer able to follow instructions.

However, as seen in FIGS. 7 and 8, (data from Trial 2, carotenoid and fish oil intervention) and Table 2, progression of AD was very markedly less, with carers reporting improved cognitive function, visual function and general wellness. Of note, no patients dropped out of Trial 2 and the comments received were as follows: memory improvement, eyesight improvement, AD didn't get worse.

### CONCLUSIONS

The examples provided here suggest that AD may be a nutrient-deficiency disease. The nutrients in question are the carotenoids (lutein and zeaxanthin) and DHA, which have been identified in the human brain. These nutrients have the potential to support brain health and reduce risk of AD via their antioxidant, anti-inflammatory, and structural roles. The inventors hypothesise that AD is a nutrient-deficiency disease, and that the positive medical responses observed in the trials are due to correction of this deficiency. However, in circumstances where the disease is developed, the correction of the nutrient deficiency is not capable to reverse the disease; but, stabilization of brain health ad function is achieved, consistent with halting AD progression in patients supplemented with carotenoids and fish oil.

The inventors suggest that AD is a deficiency disease of two nutrient components: a. the macular pigments lutein and zeaxanthin, which are obtained from eating green leafy vegetables (e.g. spinach and kale) and b. DHA, obtained from eating fatty fish (e.g. salmon).

The data shows that AD can be prevented by a satisfactory intake of, e.g. the abovementioned foods.

### Example 2

The evolution of mild cognitive impairment Mild cognitive impairment (MCI) is recognised as a deterioration in cognitive function which exceeds that anticipated for an individual based on their age and education level. However, these changes in cognition are not significant enough to impact an individual's independence or ability to perform activities of daily living (33). MCI is often, but not always, a transitional phase between the cognitive changes that one expects as one ages and very early dementia. It is difficult to diagnose and its prognosis is notoriously unpredictable. MCI was introduced as a clinical entity in 1988 (34) and was based on stage 3 of the global deterioration scale. Clinical criteria were established in the late 1990s, due to the increasing awareness of dementia, (35) and were revised in 2003 to form the core criteria for diagnosing MCI (36). The criteria for a diagnosis of MCI overlap with the diagnosis of Alzheimer's Disease (AD) where there is a subjective cognitive complaint (reported by the individual, an informant or raised by the individual's clinician), which is then confirmed using objective cognitive measures (preferably based on normative data for the individual). Importantly, a diagnosis of MCI differs from a diagnosis of AD in that individuals must be able to perform functional activities (e.g. standing, walking) independently and must have no difficulty or very mild difficulty in instrumental activities of daily living (e.g. housework, preparing meals). A final prerequisite to a MCI diagnosis is to be classified by the clinician as non-demented.

MCI can be sub-divided into amnestic or non-amnestic, depending on which cognitive domain(s) the individual experiences impairment in. In amnestic MCI, memory is the predominant problem and is associated with a higher risk of conversion to AD. Individuals with non-amnestic MCI experience impairment in cognitive domains other than memory (e.g. language, visuospatial skills) and have a higher risk of converting to other forms of dementia such as Lewy Body dementia. Both amnestic and non-amnestic MCI can be further divided into single- and multi-domain subtypes. Thus, an individual can be classified into: a) amnestic single domain; b) amnestic multiple domain; c) non-amnestic single domain; or d) non-amnestic multiple domain. Of note, other classifications of MCI have also been developed. These include: MCI due to AD, which was proposed by a workgroup formulated by the National Institute on Aging-Alzheimer's Association (37) and identifies individuals with underlying AD pathology; cognitive impairment not demented (CIND), where the cognitive functioning of individuals falls below normal but they do not fully meet MCI criteria (e.g. cognitive impairment due to delirium or chronic substance misuse) (38); and neurocognitive disorders (ND), which was developed by the American Psychiatric Association in 2013 to describe features of impairment that are not dementia and can be sub-divided into delirium, mild ND and major ND (39). The primary difference between NDs and MCI is that NDs can occur in all age groups whereas MCI typically occurs in older adults.

A Non-Pharmacological Approach to MCI

Due to the increased risk of mortality and progression to dementia, MCI is an important public health concern. Despite its complex and dynamic nature, MCI provides a unique opportunity to understand the progression of AD and its underlying pathology over a long period of time. Crucially, it offers a window of opportunity to examine the potential of preventative strategies for modifying or delaying disease progression and improving cognitive outcomes, given that many (but not all) risk factors for MCI are modifiable. Shifting focus towards preventative strategies seems prudent given that, to date, pharmacological approaches remain elusive.

Several studies have examined the role of dietary patterns, in particular the Mediterranean diet, in slowing down rates of cognitive decline (40), reducing the risk of developing MCI (41) and reducing the risk of progressing from MCI to AD (42). Dietary components including carotenoids, omega-3 fatty acids and vitamin E selectively accumulate in brain tissue where they play important physiological functions and activities (43, 44). The brain is a lipid-dense organ with large amounts of omega-3FAs (in particular, docosahexaenoic acid [DHA]) located in the phospholipids that comprise the membranes of microglia and neurons. In blood and most tissues, xanthophyll (oxygen-containing) carotenoids including lutein and zeaxanthin account for just 40% of total carotenoid concentrations; however, in the brain xanthophylls account for more than 65% of total carotenoids, possibly suggesting preferential uptake. The inventors believe that these nutritional compounds can work independently and synergistically to optimise the neurocognitive environment, primarily due to their antioxidant and anti-inflammatory properties (for a review see (45). Nutritional deficiencies have been observed in individuals with MCI and AD (46-49) in comparison to cognitively healthy individuals.

Only a small number of studies have investigated the impact of carotenoids and omega-3 fatty acids independently and, to date, none have examined the potential synergistic effects of these key nutritional components among individuals with MCI. It also remains to be determined whether any response can benefit these individuals in terms of optimising their cognitive function and/or slowing down their rate of cognitive decline. The present study is designed to investigate the impact of targeted nutritional intervention with carotenoids, omega-3 fatty acids (“FAs”) and vitamin E, in combination, on cognitive function among individuals with MCI.

Materials and Method

Study Design

The Cognitive impAiRmEnt Study (CARES) is a double-blind, placebo-controlled, randomized clinical trial investigating the impact of supplementation with xanthophyll carotenoids, fish oil and vitamin E on cognitive function among individuals with MCI. Individuals were initially identified as potentially suitable for enrolment based on a medical assessment performed by Geriatricians and Psychiatrists of Old Age in the South-East catchment area of Ireland. Both amnestic and non-amnestic MCI were included. MCI sub-type classification was not performed. Individuals with MCI were randomized to either the active intervention (a total daily dose of 10 mg lutein; 10 mg meso-zeaxanthin; 2 mg zeaxanthin; 15 mg vitamin E (alpha-tocopherol); 1 g fish oil (of which 430 mg DHA and 90 mg eicosapentaenoic acid [EPA]) or placebo (sunflower oil) group. These doses were provided via two oval capsules, each containing one half of the daily dose of the intervention or placebo substances, as appropriate. Individuals were instructed to consume two capsules per day with a meal. Frequent phone calls were made to ensure compliance. Tablet counting was also performed at follow-up. Study visits occurred at baseline and 12 months at a single site (Nutrition Research Centre Ireland [NRCI]). Intervention randomization was performed by an electronic trial management system “Trial Controller” designed by our research group. This administration system was also used to document patient information (name and contact details), support the organisation and management of capsules required for the clinical trial and assist with the scheduling of study visits.

The primary outcome measure of CARES was change in cognitive function. Secondary outcome measures included change in the following variables: macular pigment; visual function; serum carotenoid concentrations; and plasma omega-3FA concentrations. (The results from these latter parameters are omitted from the present specification for brevity). Development of AD was also recorded. A diagnosis of MCI was based on published criteria (35, 36). Specific eligibility criteria included: self or family member reported memory loss; fulfils criteria for minimal cognitive impairment; functionally independent in activities of daily living; aged 65 years and over; no rapidly progressive or fluctuating symptoms of memory loss; no established diagnosis of early dementia (consumption of cognitive enhancement therapy such as cholinesterase inhibitors or N-methyl-D-aspartate [NMDA] receptor antagonists); no stroke disease (clinical stroke or stroke on CTB); no depression (under active review); no psychiatric illness (under active review of psychotropic medications); no glaucoma (acute angle); no consumption of carotenoid supplements, (e.g. MacuHealth) fish oil supplements (e.g. Souvenaid) or cod liver oil; and no fish allergy.

MCI Screening

Prior to enrolment, all individuals that expressed an interest in participating in the trial completed a screening assessment to confirm eligibility. This included assessing cognitive function using the Repeatable Battery for the Assessment of Neuropsychological Status (RBANS) Record Form A, and the Montreal Cognitive Assessment (MoCA) version 7.1. Level of functional ability was assessed using the Bristol Activities of Daily Living Scale (BADLS) and the Alzheimer's Questionnaire (AQ). In the event where an informant was not present during the assessment, a family member or carer was contacted via telephone to complete functional ability assessments. In circumstances where no informant was available, the researcher administered the questionnaires to the patient. A brief description of each of these assessments is provided below. Individuals that fulfilled the criteria for each cognitive and functional assessment were invited to participate in the clinical trial. Individuals with borderline scores from the screening assessments were referred to a consensus panel (via email or conference call) consisting of two Consultant Geriatricians and a Clinical.

Neuropsychologist for assessment of eligibility (50, 51). Eligible individuals were then invited to enroll into the study. Prior to enrolment, written informed consent was obtained from all individuals. Ethical approval was granted by the Research Ethics Committees of the Waterford Institute of Technology and University Hospital Waterford in Waterford, Ireland. CARES (trial registration number: ISRCTN10431469) adhered to the tenets of the declaration of Helsinki and followed the full code of ethics with respect to recruitment, testing and general data protection regulations as set out by the European Parliament and Council of the European Union.

Assessing Cognitive Function

Global Cognition

The MoCA was used at the screening stage to assess global cognition. It is a short (10 minute) cognitive screening tool with high sensitivity and specificity for detecting MCI (52, 53). Thirty items assess multiple cognitive domains including visuospatial abilities, executive function, phonemic fluency, attention, immediate and delayed recall, language and orientation. In the present study, a score between 19 and 25 was desirable for enrolment. The RBANS was used to measure global cognition at screening and 12-month follow-up. Five domains of cognition (immediate memory, visuospatial/constructional, language, attention and delayed memory) were assessed using 12 sub-tests. The RBANS takes approximately 30 minutes to administer and is a core diagnostic tool for detecting and characterising dementia (54). In the present study, a score of <78 was desirable for enrolment.

Statistical Analysis

The statistical package IBM SPSS version 25 was used. Given that data in both intervention groups were not normally distributed, the small sample size, and the presence of ranked data, a non-parametric approach was taken. Results were expressed as median (inter quartile range [IQR]) for all variables. Between-group differences (i.e. active versus placebo) were analysed using Mann-Whitney U, Wilcoxon Signed Rank or Chi-square tests as appropriate. The Mann-Whitney U test was also used to examine the significance of change in nutrition variables over time between active and placebo intervention groups. Significance values were not computed to examine change in cognition or vision variables over time between both groups due to a lack of statistical power and the small magnitude of change over time observed for these variables. As an alternative, the average percentage change per subject was reported. Of note, percentage change could not be calculated for some variables (i.e. episodic memory) as baseline values were recorded as 0. Thus, the average change per subject was reported.

Results

The baseline data for demographic, health and lifestyle, cognitive function, functional ability, visual function, and biochemical data for active and placebo intervention groups were statistically comparable, with no relevant differences between the groups at baseline.

Change in Cognition Variables Over Time

Table 3 shows improvements (ranging from 6% to 18%) in global cognition in the active intervention group, where all domains of the RBANS assessment improved over the 12-month intervention period (except for the visuospatial and language domains, which both declined by 1%) (FIG. 9). RBANS results were mixed in the placebo group. There was a significant improvement in immediate memory in the active group and there was no significant change in the placebo group.

Comment

Evidence is provided that a mixture of macular carotenoids, vitamin E, and fish oil combined can improve cognitive function in patients with MCI and therefore prevent AD.

TABLE 3
Average percentage change in global cognition over 12
months between active and placebo intervention groups

| | Variable | | | | | | |
|---|---|---|---|---|---|---|---|
| | Active Intervention | | | | | | |
| | | Baseline | | 12 months | | % | |
| Global Cognition | n | Median (IQR) | | Median (IQR) | | Δ | Outcome |
| RBANS immediate | 6 | 78.0 | (73.3-82.5) | 91.0 | (81.3-100.8) | +18 | Improved |
| memory | | | | | | | |
| RBANS visuospatial | 6 | 107.0 | (101.5-110.8) | 105.0 | (101.5-112.0) | −1 | Declined |
| RBANS language | 6 | 91.0 | (88.0-96.0) | 89.0 | (88.0-93.0) | −1 | Declined |
| RBANS attention | 6 | 84.5 | (68.0-103.8) | 91.0 | (79.0-100.0) | +7 | Improved |
| RBANS delayed memory | 6 | 85.0 | (63.3-96.5) | 86.0 | (63.0-106.3) | +12 | Improved |
| RBANS total scale | 6 | 82.0 | (78.0-91.5) | 87.0 | (82.3-99.3) | +6 | Improved |

| | Variable | | | | | | |
|---|---|---|---|---|---|---|---|
| | Placebo Intervention | | | | | | |
| | | Baseline | | 12 months | | % | |
| Global Cognition | n | Median (IQR) | | (Median (IQR) | | Δ | Outcome |
| RBANS immediate | 7 | 94.0 | (85.0-100.0) | 90.0 | (81.0-103.0) | 0 | Unchanged |
| memory | | | | | | | |
| RBANS visuospatial | 7 | 96.0 | (87.0-109.0) | 96.0 | (84.0-109.0) | 0 | Unchanged |
| RBANS language | 7 | 88.0 | (82.0-92.0) | 92.0 | (72.0-105.0) | +2 | Improved |
| RBANS attention | 7 | 79.0 | (75.0-85.0) | 79.0 | (79.0-94.0) | 0 | Unchanged |
| RBANS delayed memory | 7 | 71.0 | (60.0-93.0) | 90.0 | (78.0-98.0) | +14 | Improved |
| RBANS total scale | 7 | 82.5 | (76.3-87.3) | 88.5 | (78.5-91.3) | +3 | Improved |

Data displayed are median (inter quartile range); % Δ: average percentage change per subject; RBANS: Repeatable Battery for the Assessment of Neuropsychological Status.

The content of all publications mentioned in the present specification, including but not limited to those expressly listed below, are herein incorporated by reference.

### REFERENCES

[1] Alzheimer's Association (2017) 2017 Alzheimer's disease facts and figures, Alzheimer's Association, Chicago, Ill., USA.
[2] Tangney et al., (2011) Adherence to a Mediterranean-type dietary pattern and cognitive decline in a community population. Am J Clin Nutr 93, 601-607.
[3] Feart et al., (2009) Adherence to a Mediterranean diet, cognitive decline, and risk of dementia. J.A.M.A. 302, 638-648.
[4] Trichopoulou et al., (2015) Mediterranean diet and cognitive decline over time in an elderly Mediterranean population. Eur. J Nutr. 54, 1311-1321.
[5] Pelletier et al., (2015) Mediterranean diet and preserved brain structural connectivity in older subjects. Alzheimers Dement 11, 1023-1031.
[6] McEvoy et al., (2017) Neuroprotective Diets Are Associated with Better Cognitive Function: The Health and Retirement Study. J Am Geriatr Soc 65, 1857-1862.
[7] Lourida et al., (2013) Mediterranean diet, cognitive function, and dementia: a systematic review. Epidemiology 24, 479-489.
[8] Psaltopoulou et al., (2013) Mediterranean diet, stroke, cognitive impairment, and depression: A meta-analysis. Ann Neurol 74, 580-591.
[9] Singh et al., (2014) Association of mediterranean diet with mild cognitive impairment and Alzheimer's disease: a systematic review and meta-analysis. J Alzheimers Dis 39, 271-282.
[10] Scarmeas et al., (2006) Mediterranean diet and risk for Alzheimer's disease. Ann Neurol 59, 912-921.
[11] Scarmeas et al., (2009) Mediterranean diet and mild cognitive impairment. Arch Neurol 66, 216-225.
[12] Sofi et al., (2014) Mediterranean diet and health status: an updated meta-analysis and a proposal for a literature-based adherence score. Public Health Nutr 17, 2769-2782.
[13] Muldoon et al., (2014) Long-chain omega-3 fatty acids and optimization of cognitive performance. Mil Med 179, 95-105.
[14] Cole et al., (2005) Prevention of Alzheimer's disease: Omega-3 fatty acid and phenolic anti-oxidant interventions. Neurobiol Aging 26 Suppl 1, 133-136.
[15] Fotuhi et al., (2009) Fish consumption, long-chain omega-3 fatty acids and risk of cognitive decline or Alzheimer disease: a complex association. Nat Clin Pract Neurol 5, 140-152.
[16] Thomas et al., (2015) Omega-3 Fatty Acids in Early Prevention of Inflammatory Neurodegenerative Disease: A Focus on Alzheimer's Disease. Biomed Res Int 2015, 172801.
[17] Cole & Frautschy et al., (2010) DHA may prevent age-related dementia. J Nutr 140, 869-874.
[18] Abubakari A R, Naderali M M, Naderali E K (2014) Omega-3 fatty acid supplementation and cognitive function: are smaller dosages more beneficial? Int J Gen Med 7, 463-473.
[19] Yurko-Mauro et al., (2010) Beneficial effects of docosahexaenoic acid on cognition in age-related cognitive decline. Alzheimers Dement 6, 456-464.
[20] Phillips et al., (2015) No Effect of Omega-3 Fatty Acid Supplementation on Cognition and Mood in Individuals with Cognitive Impairment and Probable Alzheimer's Disease: A Randomised Controlled Trial. Int J Mol Sci 16, 24600-24613.
[21] van de Rest et al., (2008) Effect of fish oil on cognitive performance in older subjects: a randomized, controlled trial. Neurology 71, 430-438.
[22] Perry et al., (2009) Xanthophyll (lutein, zeaxanthin) content in fruits, vegetables and corn and egg products. Journal of Food Composition and Analysis 22, 9-15.
[23] Johnson et al., (2013) Relationship between Serum and Brain Carotenoids, alpha-Tocopherol, and Retinol Concentrations and Cognitive Performance in the Oldest Old from the Georgia Centenarian Study. J Aging Res 2013, 951786.
[24] Craft et al., (2004) Carotenoid, tocopherol, and retinol concentrations in elderly human brain. J Nutr Health Aging 8, 156-162.
[25] Feart et al., (2016) Plasma Carotenoids Are Inversely Associated With Dementia Risk in an Elderly French Cohort. J Gerontol A Biol Sci Med Sci 71, 683-688.
[26] Min & Min (2014) Serum lycopene, lutein and zeaxanthin, and the risk of Alzheimer's disease mortality in older adults. Dement Geriatr Cogn Disord 37, 246-256.
[27] Loef & Walach (2012) Fruit, vegetables and prevention of cognitive decline or dementia: a systematic review of cohort studies. J Nutr Health Aging 16, 626-630.
[28] Hammond et al., (2017) Effects of Lutein/Zeaxanthin Supplementation on the Cognitive Function of Community Dwelling Older Adults: A Randomized, Double-Masked, Placebo-Controlled Trial. Front Aging Neurosci 9, 254.
[29] Johnson et al., (2008) Cognitive findings of an exploratory trial of docosahexaenoic acid and lutein supplementation in older women. Nutr Neurosci 11, 75-83.
[30] Chew et al., Age-Related Eye Disease Study 2 Research G (2015) Effect of Omega-3 Fatty Acids, Lutein/Zeaxanthin, or Other Nutrient Supplementation on Cognitive Function: The AREDS2 Randomized Clinical Trial. JAMA 314, 791-801.
[31] Mangialasche et al., (2010) High plasma levels of vitamin E forms and reduced Alzheimer's disease risk in advanced age. J Alzheimers Dis 20, 1029-1037.
[32] Livingstone et al., (2017) Dementia prevention, intervention, and care. Lancet 390, 2673-2734.
[33] Petersen R C, Roberts R O, Knopman D S, Boeve B F, Geda Y E, Ivnik R J, et al. Mild cognitive impairment: ten years later. Archives of Neurology. 2009; 66(12):1447-55.
[34] Reisberg B, Ferris S H, Kluger A, Franssen E, Wegiel J, and de Leon M J. Mild cognitive impairment (MCI): a historical perspective. International Psychogeriatrics. 2008; 20(1):18-31.
[35] Petersen R C, Smith G E, Waring S C, Ivnik R J, Tangalos E G, and Kokmen E. Mild cognitive impairment: clinical characterization and outcome. Archives of Neurology. 1999; 56(3):303-8.
[36] Winblad B, Palmer K, Kivipelto M, Jelic V, Fratiglioni L, Wahlund L O, et al. Mild cognitive impairment—beyond controversies, towards a consensus: report of the International Working Group on Mild Cognitive Impairment. Journal of Internal Medicine. 2004; 256(3):240-6.
[37] Albert M S, DeKosky S T, Dickson D, Dubois B, Feldman H H, Fox N C, et al. The diagnosis of mild cognitive impairment due to Alzheimer's disease: recommendations from the National Institute on Aging-Alzheimer's Association workgroups on diagnostic guidelines for Alzheimer's disease. Alzheimers Dement. 2011; 7(3):270-9.
[38] Roberts R, and Knopman D S. Classification and epidemiology of MCI. Clinics in Geriatric Medicine. 2013; 29(4):753-72.
[39] Assaf G, and Tanielian M. Mild cognitive impairment in primary care: a clinical review. Postgraduate Medical Journal. 2018; 94(1117):647-52.
[40] Lourida I, Soni M, Thompson-Coon J, Purandare N, Lang I A, Ukoumunne O C, et al. Mediterranean diet, cognitive function, and dementia: a systematic review. Epidemiology (Cambridge, Mass.). 2013; 24(4):479-89.
[41] Roberts R O, Geda Y E, Cerhan J R, Knopman D S, Cha R H, Christianson T J, et al. Vegetables, unsaturated fats, moderate alcohol intake, and mild cognitive impairment. Dement Geriatr Cogn Disord. 2010; 29(5):413-23.
[42] Singh B, Parsaik A K, Mielke M M, Erwin P J, Knopman D S, Petersen R C, et al. Association of mediterranean diet with mild cognitive impairment and Alzheimer's disease: a systematic review and meta-analysis. Journal of Alzheimer's disease: JAD. 2014; 39(2):271-82.
[43] Craft N E, Haitema T B, Garnett K M, Fitch K A, and Dorey C K. Carotenoid, tocopherol, and retinol concentrations in elderly human brain. J Nutr Health Aging. 2004; 8(3):156-62.
[44] Chang C Y, Ke D S, and Chen J Y. Essential fatty acids and human brain. Acta Neurol Taiwan. 2009; 18(4):231-41.
[45] Power R, Prado-Cabrero A, Mulcahy R, Howard A, and Nolan J M. The Role of Nutrition for the Aging Population: Implications for Cognition and Alzheimer's Disease. Annu Rev Food Sci Technol. 2019; 10:619-39.
[46] Gardener S, Gu Y, Rainey-Smith S R, Keogh J B, Clifton P M, Mathieson S L, et al. Adherence to a Mediterranean diet and Alzheimer's disease risk in an Australian population. Translational Psychiatry. 2012; 2:e164.
[47] Rinaldi P, Polidori M C, Metastasio A, Mariani E, Mattioli P, Cherubini A, et al. Plasma antioxidants are similarly depleted in mild cognitive impairment and in Alzheimer's disease. Neurobiol Aging. 2003; 24(7):915-9.
[48] Nolan J M, Loskutova E, Howard A N, Moran R, Mulcahy R, Stack J, et al. Macular Pigment, Visual Function, and Macular Disease among Subjects with Alzheimer's Disease: An Exploratory Study. Journal of Alzheimer's disease. 2014.
[49] Cunnane S C, Schneider J A, Tangney C, Tremblay-Mercier J, Fortier M, Bennett D A, et al. Plasma and brain fatty acid profiles in mild cognitive impairment and Alzheimer's disease. Journal of Alzheimer's disease: JAD. 2012; 29(3):691-7.
[50] Dubois B, and Albert M L. Amnestic MCI or prodromal Alzheimer's disease? The Lancet Neurology. 2004; 3(4):246-8.
[51] Albert M S, DeKosky S T, Dickson D, Dubois B, Feldman H H, Fox N C, et al. The diagnosis of mild cognitive impairment due to Alzheimer's disease: recommendations from the National Institute on Aging-Alzheimer's Association workgroups on diagnostic guidelines for Alzheimer's disease. Alzheimer's &dementia: the Journal of the Alzheimer's Association. 2011; 7(3):270-9.
[52] Nasreddine Z S, Phillips N A, Bedirian V, Charbonneau S, Whitehead V, Collin I, et al. The Montreal Cognitive Assessment, MoCA: a brief screening tool for mild cognitive impairment. Journal of the American Geriatrics Society. 2005; 53(4):695-9.
[53] Luis C A, Keegan A P, and Mullan M. Cross validation of the Montreal Cognitive Assessment in community dwelling older adults residing in the Southeastern US. Int J Geriatr Psychiatry. 2009; 24(2):197-201.
[54] Randolph C, Tierney M C, Mohr E, and Chase T N. The Repeatable Battery for the Assessment of Neuropsychological Status (RBANS): preliminary clinical validity. Journal of Clinical and Experimental Neuropsychology. 1998; 20(3):310-9.

1. A method for the prevention and/or treatment of mild cognitive impairment in a human subject; the method comprising the step of administering an effective amount of a composition comprising each of the following: lutein, zeaxanthin, meso-zeaxanthin, vitamin E, and at least one omega-3 fatty acid.

2. The method according to claim 1, wherein the said at least one omega-3 fatty acid is docosahexaenoic acid.

3. The method according to claim 1, wherein the said at least one omega-3 fatty acid is provided in the composition as a fish oil.

4. The method according to claim 1, wherein the said at least one omega-3 fatty acid comprises at least two omega-3 fatty acids.

5. The method according to claim 4, wherein the said at least two omega-3 fatty acids comprise docosahexaenoic acid and eicosapentaenoic acid.

6. The method according to claim 1, wherein the said at least one omega-3 fatty acid is provided as the free acid, as a salt or as a trigylceride.

7. The method according to claim 6, wherein the salt comprises a monocation.

8. The method according to claim 7, wherein the salt comprises a metal monocation.

9. The method according to claim 1, wherein administering the composition to a subject prevents or delays the development of the symptoms of Alzheimer's disease in the subject.

10. The method according to claim 1, wherein the composition is administered orally to the subject at least three times a week.

11. The method according to claim 1, wherein the composition is administered to the subject daily.

12. The method according to claim 11, wherein the daily consumption of the composition provides 10 mgs meso-zeaxanthin, 10 mgs lutein, 2 mgs zeaxanthin, 15 mgs of vitamin E and 1 gram of fish oil.

13. The method according to claim 12, wherein the daily consumption is provided by two capsules or other oral dosage forms.

14. The method according to claim 1, wherein the composition further comprises one or more of the following: acidity regulators; anticaking agents selected from the group consisting of sodium aluminosilicate, calcium or magnesium carbonate, calcium silicate, sodium or potassium ferricyanide; antioxidants, selected from the group consisting of vitamin C and polyphenols; colorings selected from the group consisting of artificial colorings FD&C Blue No. 1, Blue No. 2, Green No. 3, Red No. 40, Red No. 3, Yellow No. 5 and Yellow No. 6; and the natural colorings caramel, annatto, cochineal, betanin, turmeric, saffron, paprika; color retention agents; emulsifiers; flavours; flavour enhancers; preservatives; stabilizers; sweeteners and thickeners.

15. The method according to claim 1, wherein the composition is in the form of a tablet or capsule.

16. The method according to claim 1, wherein the subject consumes the composition on a daily basis over a period of at least 12 months.

17. The method according to claim 1, wherein the subject already exhibits one or more symptoms of Alzheimer's disease prior to first administration of the composition.

18. The method according to claim 1, wherein the composition is first administered to the subject before the subject exhibits any symptoms of Alzheimer's disease.

19. The method according to claim 1, comprising use of a composition substantially consisting of lutein, zeaxanthin, meso-zeaxanthin, at least one omega-3 poly-unsaturated fatty acid or corresponding salt or triglyceride, vitamin E, and a diluent, carrier or excipient.

20. The method according to claim 19, wherein the omega-3 poly-unsaturated fatty acid or corresponding salt or triglyceride is DHA or a salt or triglyceride of DHA.

21. The method according to claim 1, wherein the method comprises daily oral administration of a unit dose form composition comprising each of: vitamin E; lutein; zeaxanthin; meso-zeaxanthin; and DHA and/or EPA, in preventing or retarding the progression of cognitive impairment in a subject already exhibiting one or more symptoms of mild cognitive impairment prior to administration of the composition.

22. The method of claim 1, wherein performance of the method results in an improvement in the subject's RBANS immediate memory score of at least 5% relative to the subject's RBANS immediate memory score at the commencement of the method.
